(19) United States
(12) Patent Application Publication
(10) Pub. No.: US 2007/0292969 A1
(43) Pub. Date: Dec. 20, 2007

(54) Pregnancy test device

(76) Inventor: Armando Canseco, San Antonio, TX (US)

Correspondence Address: NATIONAL IP RIGHTS CENTER, LLC;SCOTT J. FIELDS, ESQ., 550 TOWNSHIP LINE ROAD, SUITE 400, BLUE BELL, PA, 19422, US

(21) Appl. No.: 11/454,642

(22) Filed: Jun. 15, 2006

(51) Int. Cl. G01N 33/53

(52) U.S. Cl. 436/510

(57) ABSTRACT

A pregnancy test device aids a woman in determining whether or not she is pregnant. The pregnancy test device includes a molded body, having a series of apertures and a display window; a sample pad, disposed within the molded body for receiving a urine sample, said sample pad treated to detect the presence of human chorionic gonadotropin or its β-subunit, and exposed through the series of apertures in the molded body; a membrane, operatively connected to the sample pad, to display the results of the pregnancy test, and said membrane positioned to be viewed through the display window of the molded body; and gold, disposed between the sample pad and the membrane.

### FIELD OF THE INVENTION

The present invention relates to testing devices, and in particular to a pregnancy test device.

### BACKGROUND OF THE INVENTION

At home pregnancy tests are used more often today to allow a couple to determine whether or not they will be parents before the need to go to the doctor for verification. As more and more information becomes available about diets and behaviors that could be harmful to a child in utero, mothers have become more concerned about knowing when they may be pregnant in earlier stages so that they may curb their diet and behavior to a healthier lifestyle for their child. A number of pregnancy tests have been introduced to the market to allow a mother to test whether or not she is pregnant.

U.S. Pat. No. 4,033,723 discloses a method and device for detecting pregnancy. The test involves concentration by ultrafiltration of a sample of urine or serum from a subject; followed by determining the presence of human chorionic gonadotropin or of its .beta.-subunit in the concentrated sample.

U.S. Pat. No. 4,123,509 discloses a method and device for detecting pregnancy. The test involves concentration by ultrafiltration of a sample of urine or serum from a subject; followed by determining the presence of human chorionic gonadotropin or of its .beta.-subunit in the concentrated sample.

Current testing devices provide for a woman to void on a wick that transfers the urine to a sample pad which reacts with the urine to determine the presence of Human Chorionic Gonadotropin (HCG). This increases the amount of time the test takes and the overall cost of the device. It would therefore be advantageous if a pregnancy device existed that allowed for the woman to void directly onto the sample pad, while protecting the sample pad itself, and providing faster results.

### SUMMARY OF THE INVENTION

It is an aspect of the present invention to provide a pregnancy test device with rapid results.

It is a further aspect of the present invention to provide a pregnancy test device having a molded body, having a series of apertures and a display window; a sample pad, disposed within the molded body for receiving a urine sample, said sample pad treated to detect the presence of human chorionic gonadotropin or its β-subunit, and exposed through the series of apertures in the molded body; a membrane, operatively connected to the sample pad, to display the results of the pregnancy test, and said membrane positioned to be viewed through the display window of the molded body; and gold, disposed between the sample pad and the membrane.

It is yet a further aspect of the present invention to provide a pregnancy test having a molded body, having a first end and second end, formed of a base and a cover fastened to the base; a display disposed generally centrally on the cover of the molded body, and a series of apertures formed into the cover proximate to the second end of the cover of the molded body; a sample pad, disposed within the base of the molded body beneath the apertures in the cover of the molded body, for receiving a urine sample, said sample pad treated to detect the presence of human chorionic gonadotropin or its β-subunit, and exposed through the series of apertures in the cover of the molded body; a membrane, disposed within the base of the molded body, operatively connected to the sample pad, to display the results of the pregnancy test, said membrane positioned to be viewed through the display window of the cover of the molded body; and gold, mounted within the base of the molded body, disposed between the sample pad and the membrane.

In accordance with a first aspect of the present invention, a novel pregnancy test device is provided. The novel pregnancy test device includes a molded body, having a series of apertures and a display window; a sample pad, disposed within the molded body for receiving a urine sample, said sample pad treated to detect the presence of human chorionic gonadotropin or its β-subunit, and exposed through the series of apertures in the molded body; a membrane, operatively connected to the sample pad, to display the results of the pregnancy test, and said membrane positioned to be viewed through the display window of the molded body; and gold, disposed between the sample pad and the membrane.

In accordance with another aspect of the present invention, a novel pregnancy test device is provided. The novel pregnancy test device includes a pregnancy test having a molded body, having a first end and second end, formed of a base and a cover fastened to the base; a display disposed generally centrally on the cover of the molded body, and a series of apertures formed into the cover proximate to the second end of the cover of the molded body; a sample pad, disposed within the base of the molded body beneath the apertures in the cover of the molded body, for receiving a urine sample, said sample pad treated to detect the presence of human chorionic gonadotropin or its β-subunit, and exposed through the series of apertures in the cover of the molded body; a membrane, disposed within the base of the molded body, operatively connected to the sample pad, to display the results of the pregnancy test, said membrane positioned to be viewed through the display window of the cover of the molded body; and gold, mounted within the base of the molded body, disposed between the sample pad and the membrane.

### BRIEF DESCRIPTION OF THE DRAWINGS

The foregoing summary, as well as the following detailed description of a preferred embodiment of the present invention will be better understood when read with reference to the appended drawings, wherein:

FIG. 1a is a top plan view of a prior art pregnancy test device.

FIG. 1b is a top plan view of the prior art pregnancy test device of FIG. 1a with the cover removed.

FIG. 2a is a top plan view of a pregnancy test device in accordance with the present invention.

FIG. 2b is a top plan view of the top plan view of the pregnancy test device of FIG. 2a with the cover removed.

### DETAILED DESCRIPTION OF THE PREFERRED EMBODIMENT

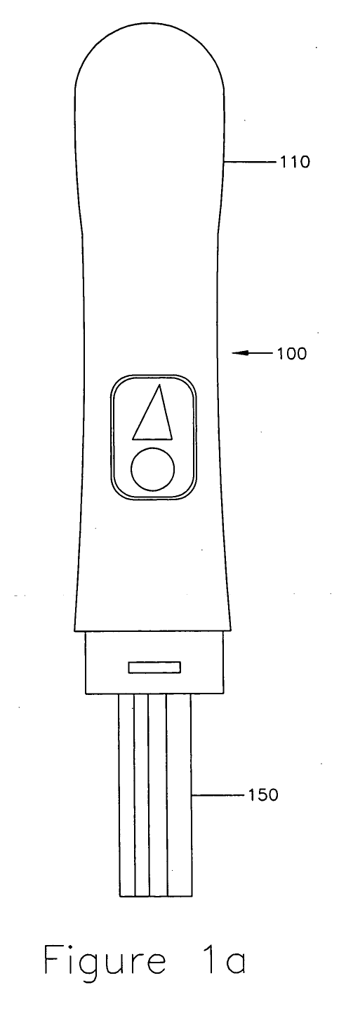

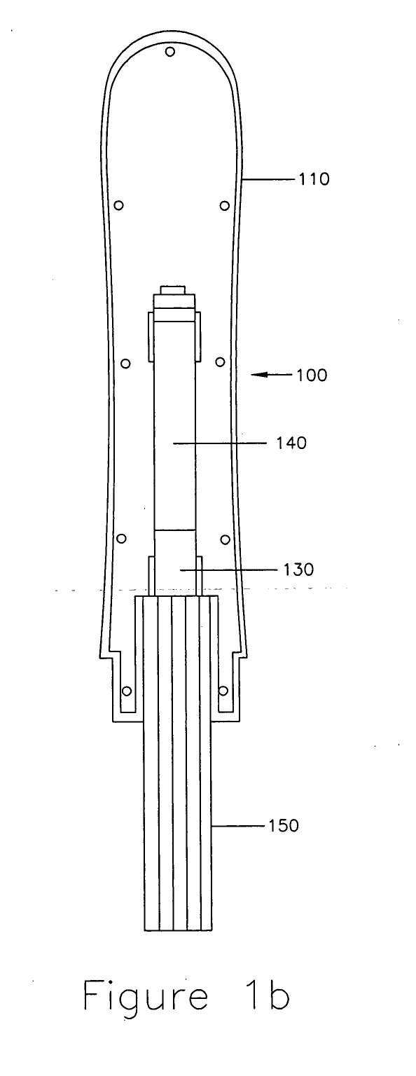

Referring now to the drawings, wherein like reference numerals refer to the same components across the several views and in particular to FIGS. 1a and 1b, a typical prior art pregnancy test device 100 is depicted. The prior art pregnancy device 100 includes a case 110. Inside of the case 110 of the prior art pregnancy device 100 is a sample pad 130. A wick 150 is mounted within the case 110 and proceed outside of the case 110 of the prior art pregnancy device 100. The wick 150 is in physical communication with the sample pad 130. A coated membrane 140 is disposed within the case 110 and in physical communication with the sample pad 130. The coated membrane 140 displays the results of a pregnancy test.

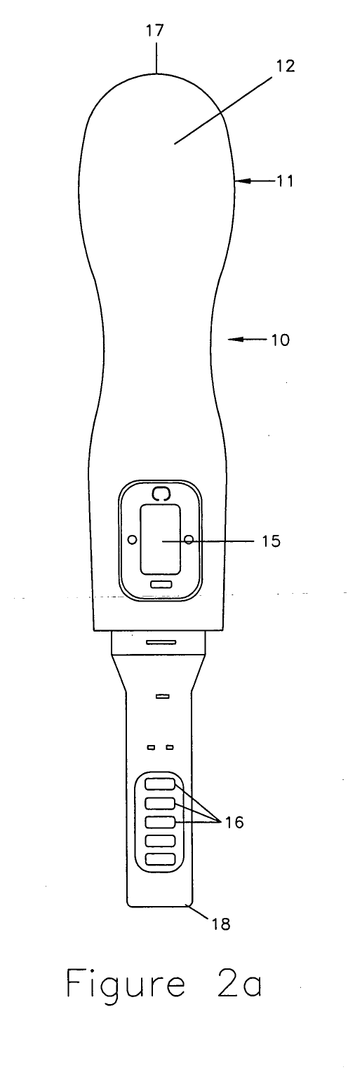

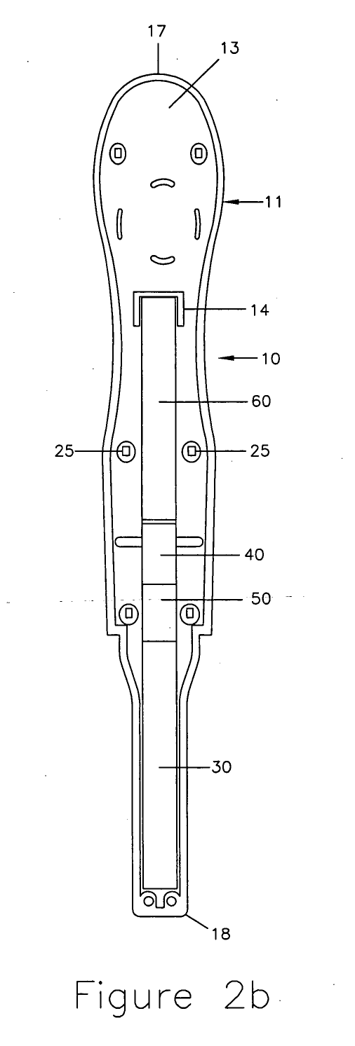

In FIGS. 2a and 2b a pregnancy test device 10 is shown. The pregnancy test device 10 includes a molded body 11, a display 15, and apertures 16. The molded body 11 of the pregnancy test device 10 includes a first end 17 and a second end 18. The molded body 11 is formed of a cover 12 and a base 13.

The cover 12 of the molded body 11 includes the display 15 generally centrally between the first end 17 of the molded body and the second end 18 of the molded body. The display 15 shows the results of a pregnancy test. The apertures 16 are disposed proximate to the second end 18 of the cover 13. In a preferred embodiment of the present invention, the apertures 16 are formed only in the cover 13 of the molded body 11; however, the apertures 16 may be placed anywhere on the molded body 11 known to one of ordinary skill in the art.

FIG. 2b depicts the base 13 of the molded body 11. A series of mounts 25 are formed into the base 13 of the molded body 11 to receive the cover 12. The cover 12 is fastened to the base 13 at the mounts 25 by any means known to one of ordinary skill in the art. In a preferred embodiment, the fasteners 25 may be snap-fit clips to snap-fit the cover 12 to the base 13.

A sample pad 30 is disposed in the base 13 of the molded body 11 proximate to the second end 18 of the molded body 11 generally beneath the apertures 16 formed into the cover 12 of the molded body 11. In this way, a urine sample is transferred directly through the apertures 16 to the sample pad 30. The sample pad 30 is treated with a chemical to interact with the urine and to detect the presence of human chorionic gonadotropin (HCG) or its β-subunit. In a preferred embodiment of the present invention, the HCG reacts with monoclonal antibodies to determine the presence of the HCG.

A membrane 40 is disposed generally centrally in the base 13 of the molded body 11 and is generally under the display 15 of the cover 12. In this manner, the results on the membrane 40 are shown to the user via the display 15. A gold layer 50 is disposed between the sample pad 30 and the membrane 40. A top pad 60 is in physical communication with the membrane 40 In a preferred embodiment of the present invention, the user voids directly through the apertures 16 onto the sample pad 30. In this manner, the sample pad 30 immediately reacts with the urine to determine the presence of HCG or its β-subunit. The membrane 40 then displays the results virtually immediately through the display 15. The results could be displayed in a variety of forms known to one of ordinary skill in the art. For example, the results could be displayed as a single control line if the pregnancy test is negative and a control line and positive line if the pregnancy test is positive. However, any display method known to one of ordinary skill in the art can be employed to display a positive or negative pregnancy test through the display 15. Critically, the pregnancy test device 10 does not include a wick, such as the wick 150 in the prior art pregnancy device 100. This permits a much more rapid and accurate response since the urine sample of the user is directly voided onto the sample pad 30.

In view of the foregoing disclosure, some advantages of the present invention can be seen. For example, a novel pregnancy test device is disclosed. The novel pregnancy test operates without the use of a wick and therefore provides a more accurate and rapid response to a urine sample.

While the preferred embodiment of the present invention has been described and illustrated, modifications may be made by one of ordinary skill in the art without departing from the scope and spirit of the invention as defined in the appended claims. For example, in a preferred embodiment of the present invention, the molded body is formed of high impact plastic; however any material known to one of ordinary skill in the art may be utilized to form the molded body. Additionally, the molded body in a preferred embodiment is constructed of a base and a cover fastened together; however the molded body could be formed differently as one of ordinary skill in the art might choose to form the molded body.

What is claimed is:

1. A pregnancy test device, comprising:
a molded body, having a series of apertures and a display window;
a sample pad, disposed within the molded body for receiving a urine sample, said sample pad treated to detect the presence of human chorionic gonadotropin or its β-subunit, and exposed through the series of apertures in the molded body;
a membrane, operatively connected to the sample pad, to display the results of the pregnancy test, and said membrane positioned to be viewed through the display window of the molded body; and
gold, disposed between the sample pad and the membrane.

2. The pregnancy test device of claim 1, wherein the molded body further comprises a base and a cover fastened to the base.

3. The pregnancy test device of claim 2, wherein the molded body further comprises a first and second end, and the series of apertures are located proximate to the second end of the cover of the molded body.

4. The pregnancy test device of claim 1, wherein the sample pad is treated with monoclonal antibodies to detect the presence of human chorionic gonadotropin or its β-subunit.

5. The pregnancy test of claim 4, wherein the display shows only a control line to indicate a negative test, and an additional line to indicate a positive test.

6. The pregnancy test of claim 1, wherein the molded body is formed of high impact plastic.

7. A pregnancy test device, comprising:
a molded body, having a first end and second end, formed of a base and a cover fastened to the base;
a display disposed generally centrally on the cover of the molded body, and a series of apertures formed into the cover proximate to the second end of the cover of the molded body;
a sample pad, disposed within the base of the molded body beneath the apertures in the cover of the molded body, for receiving a urine sample, said sample pad treated to detect the presence of human chorionic gonadotropin or its β-subunit, and exposed through the series of apertures in the cover of the molded body;
a membrane, disposed within the base of the molded body, operatively connected to the sample pad, to display the results of the pregnancy test, said membrane positioned to be viewed through the display window of the cover of the molded body; and
gold, mounted within the base of the molded body, disposed between the sample pad and the membrane.

8. The pregnancy test device of claim 7, wherein the sample pad includes monoclonal antibodies to detect the presence of human chorionic gonadotropin or its β-subunit.

9. The pregnancy test device of claim 8, wherein the display displays only a control line to indicate a negative test, and an additional line to indicate a positive test.

10. The pregnancy test of claim 7, wherein the molded body is formed of high impact plastic.
